(19) United States
(12) Patent Application Publication
(10) Pub. No.: US 2022/0386994 A1
(43) Pub. Date: Dec. 8, 2022

(54) MEDICAL IMAGING DEVICE TO SYSTEM CONNECTION

(71) Applicant: KONINKLIJKE PHILIPS N.V., EINDHOVEN (NL)

(72) Inventors: Zan Chishti, Andover, MA (US); Hoi-Cheong Steve Sun, Lexington, MA (US)

(21) Appl. No.: 17/770,126

(22) PCT Filed: Oct. 22, 2020
(86) PCT No.: PCT/EP2020/079675

§ 371 Date: Apr. 19, 2022

Related U.S. Application Data
(60) Provisional application No. 63/082,702, filed on Sep. 24, 2020.
(60) Provisional application No. 62/926,116, filed on Oct. 25, 2019.

(51) Int. Cl. A61B 8/00

(52) U.S. Cl. CPC A61B 8/4488; A61B 8/4427; A61B 8/445

(57) ABSTRACT

An ultrasound imaging system includes an ultrasound probe comprising an ultrasound transducer array. The ultrasound imaging system further includes a processor circuit configured for communication with the ultrasound probe via a first conductive pathway and a second conductive pathway. The ultrasound imaging system further includes a first connector and a second connector configured to be selectively engaged to establish the communication between the ultrasound probe and the processor circuit. The processor circuit is configured to detect an electrical conductance along the first conductive pathway. The processor circuit is further configured to transmit data to the ultrasound probe via the second conductive pathway only after detecting the electrical conductance along the first conductive pathway.

### TECHNICAL FIELD

The present disclosure relates generally to medical imaging systems and, in particular, to connectors for imaging devices, such as ultrasound probes, imaging catheters, and the like, to interface with a processing system (e.g., a console).

### INTRODUCTION

Medical imaging devices, such as hand-held ultrasound probes and intraluminal imaging devices, may include cabling that terminates in a connector for coupling to a processing system or console. For example, the connector may mate with a corresponding connector of the console at a connection junction. Upon connection between the medical imaging device and the console, the imaging device may be operated via the console, and data generated from the imaging device may be transferred to the console. The console may then process, store, display, and/or manipulate the imaging data.

In some cases, operation of the imaging device and transmission of data between the medical imaging device and the console may begin when an improper or incomplete (e.g., partial) connection is formed between the medical imaging device and the console. As a result, the medical imaging device may overheat, malfunction, or may require reconnection with the console. For example, the console may power the imaging device via conductive pathways coupled to the connectors. The connectors may additionally couple to conductive pathways for control signals and data signals. The timing of the connection of each conductive pathway, including the power pathways, at the connectors may depend on mechanical coupling of the connectors, which may vary based on user operation. In some instances, the conductive pathways for the control signals and data signals may be coupled at the connectors before the conductive pathways for power, resulting in only a partial connection between the connectors. In this state, if operation of the imaging device and/or data transmission between the imaging device and the console is initiated before the conductive pathways corresponding to power are mated at the connectors (e.g., before the medical imaging device is powered), data may be lost, which may cause the medical imaging device and/or the console to malfunction.

### SUMMARY

Embodiments of the present disclosure are systems, devices, and methods for a more reliable connection (e.g., electrical connection) between an ultrasound imaging device and a processing system (e.g., a console). More specifically, a connector that interfaces the ultrasound imaging device to the processing system may include one or more conductive members (e.g., electrical pins and/or electrical pads) designed to indicate successful electrical coupling between the ultrasound imaging device and the processing system. For example, the connector may include a shortened conductive member, a conductive member positioned offset relative to other conductive members in the connector, an impedance element coupled to a conductive member, or a combination thereof. By utilizing the conductive member designed to indicate successful electrical coupling between the ultrasound imaging device and the processing system, device overheating, malfunctions, and cases where reconnection between the ultrasound imaging device and the processing system is needed may be reduced. This can improve the efficiency and effectiveness of imaging procedures, which can improve patient comfort, shorten procedure times, improve diagnoses, and/or improve patient outcomes.

In some aspects, an ultrasound imaging system is provided by the present disclosure. The ultrasound imaging system can include an ultrasound probe comprising an ultrasound transducer array. The ultrasound imaging system can further include a processor circuit configured for communication with the ultrasound probe via a first conductive pathway and a second conductive pathway. The ultrasound imaging system can further include a first connector and a second connector configured to be selectively engaged to establish the communication between the ultrasound probe and the processor circuit. The processor circuit can be configured to detect an electrical conductance along the first conductive pathway and transmit data to the ultrasound probe via a second conductive pathway only after detecting the electrical conductance along the first conductive pathway.

In some aspects, the first conductive pathway can include a first conductive member of the first connector and a first conductive member of the second connector, and the second conductive pathway can include a second conductive member of the first connector and a second conductive member of the second connector. A first length of the first conductive member of the first connector and a second length of the second conductive member of the first connector can be different. In some aspects, the first length can be less than the second length. In some aspects, the first conductive member of the first connector can be offset along a lateral axis of the first connector relative to the second conductive member of the first connector. In some aspects, the first conductive pathway can include an impedance element configured to delay the processor circuit detecting the electrical conductance along the first conductive pathway. The first connector can include the impedance element. In some aspects, the second connector can include the impedance element. In some aspects, the processor circuit can be further configured for communication with the ultrasound probe via a third conductive pathway. The third conductive pathway can include a third conductive member of the first connector and a third conductive member of the second connector. The first conductive member of the first connector can be disposed at a first end of the first connector, and the third conductive member of the first connector can disposed at an opposite, second end of the first connector. The first conductive member of the second connector can disposed at a first end of the second connector, and the third conductive member of the second connector can be disposed at an opposite, second end of the second connector. In some aspects, the processor circuit can further configured to detect an electrical conductance along the third conductive pathway. The processor circuit may further be configured to transmit the data to the ultrasound probe via the second conductive pathway only after detecting the electrical conductance along the first conductive pathway and the third conductive pathway. In some aspects, the processor circuit can be further configured for communication with the ultrasound probe via a third conductive pathway, and the processor circuit can be further configured to, prior to detecting the electrical conductance along the first conductive pathway: detect an electrical conductance along the second conductive pathway; detect an electrical conductance along the third conductive pathway; and determine a first time between the detection of the electrical conductance along the second conductive pathway and the detection of the electrical conductance along the third conductive pathway. The processor circuit can be further configured to output an alert in response to a second time between the detection of the electrical conductance along a third conductive pathway and the detection of the electrical conductance along the first conductive pathway exceeding the first time. In some aspects, the third conductive pathway can include a third conductive member of the first connector and a third conductive member of the second connector. The first conductive member of the first connector can include a first length, the second conductive member of the first connector can include a second length, and the third conductive member of the first connector can include a third length. The second length can be greater than the third length, and the third length can be greater than the first length. In some aspects, the processor circuit can be configured to monitor the electrical conductance along the first conductive pathway in response to detecting an electrical conductance along the second conductive pathway. In some aspects, the processor circuit can be further configured to: determine a time between the detection of the electrical conductance along the first conductive pathway and the detection of an electrical conductance along the second conductive pathway; and output an alert if the time exceeds a threshold. In some aspects, the ultrasound imaging system can further include a cable extending between the ultrasound probe and the first connector. The ultrasound imaging system can further include a console comprising the processor circuit and the second connector. In some aspects, the ultrasound imaging system can further include a console comprising the processor circuit, a first cable extending between the ultrasound probe and the first connector, and a second cable extending between the console and the second connector. In some aspects, the ultrasound imaging system can further include an integrated circuit in communication with the ultrasound transducer array. The processor circuit can be configured to transmit the data along the second conductive pathway to the integrated circuit.

In some aspects, an ultrasound system includes an ultrasound probe comprising an ultrasound transducer array. The ultrasound system can further include a first connector electrically coupled to the ultrasound probe. The first connector can include a first connector body having a first end portion, a first conductive member coupled to the first connector body and spaced from the first end portion by a first distance, and a second conductive member coupled to the first connector body and spaced from the first end portion by a second distance different than the first distance. The ultrasound system can further include a second connector configured for mechanical and electrical coupling to the first connector. The second connector can include a second connector body having a second end portion and a third conductive member coupled to the second connector body and spaced from the second end portion by a third distance. The third conductive member can be configured to be electrically coupled to the first conductive member of the first connector. The second connector can further include a fourth conductive member coupled to the second connector body and spaced from the second end portion by a fourth distance different than the third distance. The fourth conductive member can be configured to be electrically coupled to the second conductive member of the first connector. Further, electrical coupling of the first conductive member of the first connector and the third conductive member of the second connector can indicate that the second conductive member and the fourth conductive member are coupled electrically.

In some aspects, a first length of the first conductive member of the first connector and a second length of the second conductive member of the first connector can be different. In some aspects, the first connector can further include an impedance element electrically coupled to the first conductive member.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a schematic diagram of an imaging system according to embodiments of the present disclosure.

FIG. 2 is a schematic diagram of a catheter according to embodiments of the present disclosure.

FIG. 3 is a perspective view of an imaging assembly according to embodiments of the present disclosure.

FIG. 4 is a block diagram of an imaging system according to embodiments of the present disclosure.

FIG. 5 is a schematic diagram of a processor circuit according to embodiments of the present disclosure.

FIG. 6A is a schematic diagram of a female connector and a male connector spaced from one another according to embodiments of the present disclosure.

FIG. 6B is a schematic diagram of a partially coupled female connector and a male connector according to embodiments of the present disclosure.

FIG. 7A is a schematic diagram of a female connector spaced from a male connector with a shortened connection pin according to embodiments of the present disclosure.

FIG. 7B is a schematic diagram of a female connector partially coupled to a male connector with a shortened connection pin according to embodiments of the present disclosure.

FIG. 7C is a schematic diagram of a female connector fully coupled to a male connector with a shortened connection pin according to embodiments of the present disclosure.

FIG. 8 is a timing diagram of electrical coupling between a female connector and a male connector with a shortened connection pin according to embodiments of the present disclosure.

FIG. 9 is a schematic diagram of a female connector having an impedance element coupled to a connection pad and a male connector having an impedance element coupled to a connection pin according to embodiments of the present disclosure.

FIG. 10 is a timing diagram of electrical coupling between a female connector and a male connector where an impedance element is coupled to one or both of a connection pad or a connection pin according to embodiments of the present disclosure.

FIG. 11 is a schematic diagram of a female connector and a male connector having a connection pin and an additional connection pin according to embodiments of the present disclosure.

FIG. 12 is a timing diagram of electrical coupling of a female connector and a male connector having a connection pin and an additional connection pin according to embodiments of the present disclosure.

FIG. 13 is a schematic diagram of a female connector and a male connector having a connection pin and an additional connection pin spaced from one another according to embodiments of the present disclosure.

FIG. 14 is a timing diagram of electrical coupling between a female connector and a male connector having a connection pin and an additional connection pin spaced from one another according to embodiments of the present disclosure.

FIG. 15 is a schematic diagram of a male connector spaced from a female connector having a connection pin offset from an edge of the female connector according to embodiments of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

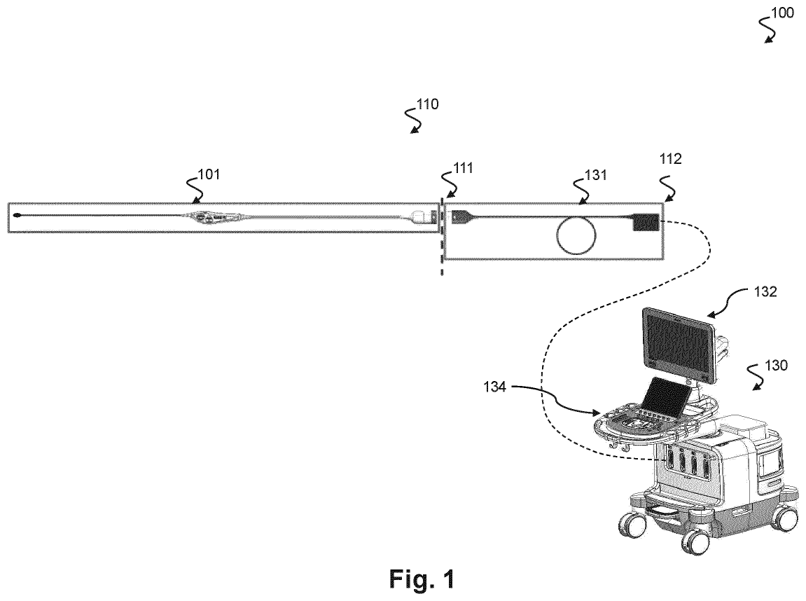

FIG. 1 is a schematic diagram of an imaging system 100 according to embodiments of the present disclosure. The system 100 may include an ultrasound imaging device 110 (e.g., an intraluminal ultrasound imaging device), a control and processing system 130 (for example, a console including a computer), and a patient interface module (PIM) 131 extending between the device 110 and the control and processing system 130.

The system 100 can be referenced as an imaging system, ultrasound imaging system, external ultrasound imaging system, intraluminal imaging system, and/or combinations thereof. Further, while some embodiments of the present disclosure refer to an imaging device, an ultrasound imaging device, or an intraluminal imaging device, it is understood that the ultrasound imaging device 110 and the system 100 generally may be used to image vessels, structures, lumens, and/or any suitable anatomy/tissue within a body of a patient including any number of anatomical locations and tissue types, including without limitation, organs including the liver, heart, kidneys, gall bladder, pancreas, lungs; ducts; intestines; nervous system structures including the brain, dural sac, spinal cord and peripheral nerves; the urinary tract; as well as valves within the blood, chambers or other parts of the heart, and/or other systems of the body. In addition to natural structures, the imaging device 110 may be used to examine man-made structures such as, but without limitation, heart valves, stents, shunts, filters and other devices. For example, the ultrasound imaging device 110 can be positioned within fluid filled or surrounded structures, both natural and man-made, such as within a body of a patient. The vessels, structures, lumens, and anatomy/tissue can include a blood vessel, as an artery or a vein of a patient's vascular system, including cardiac vasculature, peripheral vasculature, neural vasculature, renal vasculature, and/or any suitable lumen inside the body. Alternatively, the ultrasound imaging device 110 may include a hand-held ultrasound probe, a patch-based ultrasound probe, or the like, and may be used external to the body of the patient to image structures within the body.

The ultrasound imaging device 110 is contemplated as any suitable intraluminal imaging device, such as an intra-cardiac echocardiography (ICE) catheter, an intravascular ultrasound (IVUS) device, an optical coherence tomography (OCT) device, an intracardiac echocardiography (ICE) device, a transesophageal echocardiography (TEE) device, an intravascular photoacoustic (IVPA) imaging device, and/or any suitable internal imaging device. Intraluminal devices with flexible elongate members such as catheters, guide wires, and/or guide catheter are contemplated. In some embodiments, the ultrasound imaging device 110 is contemplated as an external imaging device, such as an external ultrasound probe, a patch-based ultrasound probe, and/or the like.

The PIM 131 may provide a physical and electrical connection between the ultrasound imaging device 110 and the control and processing system 130. Some embodiments of the present disclosure omit the PIM 131. In other embodiments, the PIM 131 is communicatively interposed between the ultrasound imaging device 110 and the processing system 130. In some instances, the PIM 131 can be referenced as a patient interface cable. For example, a proximal connector of the ultrasound imaging device 110, a distal connector of the PIM, and/or a proximal connector of the PIM may be configured to couple the ultrasound imaging device 110, the PIM 131, and the control and processing system together mechanically and electrically. The system 100 may include a connector junction 111 comprising a proximal connector of the ultrasound imaging device 110 and the distal connector of the PIM 131. The system 100 may include an additional connector junction 112 comprising a proximal connector of the PIM 131 and a connector of the control and processing system 130.

In some embodiments, the control and processing system 130 may include one or more computers, processors, computer systems, memory, one or more input devices, such as keyboards and any suitable command control interface device. The control and processing system 130 may be used for processing, storing, analyzing, and manipulating data, and the monitor 132 (e.g., display) may be used for displaying obtained signals generated by the imaging assembly 102. The control and processing system 130 may also be referred to as a console. In some embodiments, the PIM 131 is in mechanical and electrical communication with the control and processing system 130, such that the electrical signals are transmitted from the ultrasound imaging device 110 through the PIM 131 and to the control and processing system 130. The control and processing system 130 may include one or more processors and/or memory modules forming a processing circuit that may process the electrical signals and output a graphical representation of the imaging data on the monitor 132. One or more electrical conductors of the ultrasound imaging device 110 and PIM 131 may facilitate communication between the control and processing system 130 and the ultrasound imaging device 110. For example, a user of the control and processing system 130 may control imaging using the ultrasound imaging device 110 via a control interface 134 of the control and processing system 130. Electrical signals representative of commands from the control and processing system 130 may be transmitted to the ultrasound imaging device 110 via connectors and/or cables in the PIM 131 and the ultrasound imaging device 110. The control and processing system 130 may be transportable and may include wheels or other devices to facilitate easy transportation by a user. The control and processing system 130 may be operable to facilitate the features of the intraluminal imaging system 100 described herein. For example, a processor can execute computer readable instructions stored on the non-transitory tangible computer readable medium. The monitor 132 may be any suitable display device, such as liquid-crystal display (LCD) panel or the like.

In some embodiments, the one or more components of the ultrasound imaging device 110 may be disposable components. For example, a user, such as a physician, may obtain the catheter 101 and/or the ultrasound imaging device 110 in a sterilized packaging. In some embodiments, the ultrasound imaging device 110 may be disposed after a single use. In other embodiments, the ultrasound imaging device 110 can be sterilized and/or re-processed for more than one use. The PIM 131 may be a re-usable component that is used in multiple procedures. For example, the PIM 131 can be cleaned between individual procedures, such as being treated with disinfectants to kill bacteria. In some embodiments, the PIM 131 may not be required to be sterilized before a medical procedure. For example, the PIM 131 can be sufficiently spaced from the patient such that use of a non-sterile PIM 131 is safe for the patient. The sterile-nonsterile connection at the connector junction 111 between the ultrasound imaging device 110 and the PIM 131 may allow for a safe operating environment while saving costs by allowing expensing equipment to be reused.

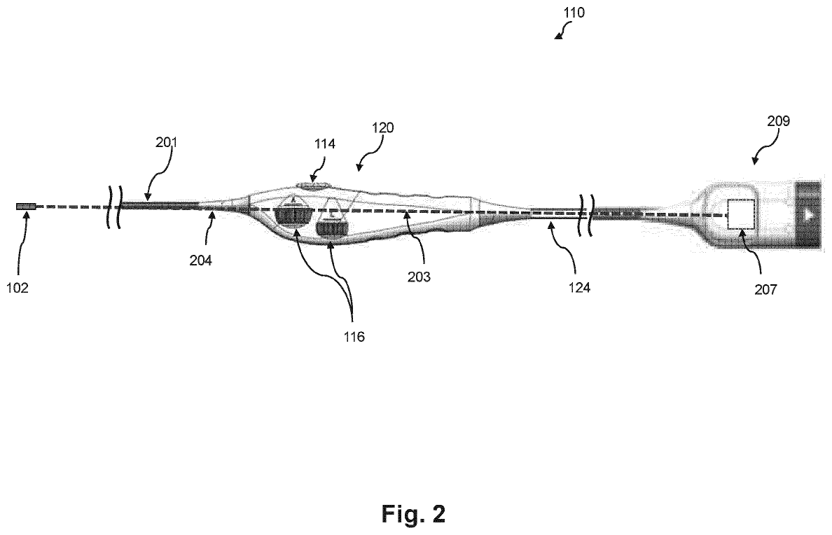

Turning now to FIG. 2, the ultrasound imaging device 110 may include a catheter 101. The catheter 101 may include one or more flexible elongate members sized and shaped, structurally arranged, and/or otherwise configured to be positioned within a body lumen of a patient. In some embodiments, the catheter 101 includes an ultrasound imaging assembly 102, a catheter body or shaft 201, a catheter cable 203, a handle 120, a conduit 124, a connector 209, and one or more printed circuit board assemblies (PCBAs) 207. The catheter cable 203 may have a small diameter configuration and a low profile that is sized to be passed or snaked through a catheter shaft 201, the handle 120, and/or the conduit 124. The cable 203 may be electrically and/or mechanically coupled to the ultrasound imaging assembly 102 at the distal portion of the catheter shaft 201, as well as the PCBA 207 at the proximal portion of the catheter 101.

In some embodiments, one or both of the catheter body/shaft 201 and catheter cable 203 may be referred to as a flexible elongate member. The catheter shaft 201 is sized and shaped, structurally arranged and/or otherwise configured to be positioned within a body lumen of a patient (e.g., vasculature such as blood vessels or chambers of the heart). Respective portions of the catheter cable 203 extend within the catheter shaft 201, the handle 120, the conduit 124, and the connector 209. The imaging assembly 102 may be attached to a distal end of the catheter shaft 201. The catheter shaft 201 may include a lumen that the catheter cable 203 may pass through. The proximal end 204 of the catheter shaft 201 may be attached to the handle 120, for example, by a resilient strain reliever. The handle 120 may be used for manipulation of the ultrasound imaging device 110 and manual control of the ultrasound imaging device 110. The ultrasound imaging device 110 may include an imaging assembly 102 with ultrasound transducer elements and associated circuitry. The handle 120 may include actuators 116, a clutch 114, and other steering control components for steering the ultrasound imaging device 110. The steering may include deflecting the distal end of the catheter cable 203.

The catheter cable 203 may pass through one or more of the catheter shaft 201, handle 120, conduit 124, and connector 209. In some embodiments, during assembly, the catheter cable 203 is sneaked through a lumen within the catheter body 201, handle 120, and conduit 124. In some embodiments, the conduit 124 is a component distinct from the cable 203. For example, the conduit can be a tubing within which the cable 203 extends. In other embodiments, the conduit 124 can be a coating defining an exterior surface of the cable 203. The coating can strengthen the cable 203 for exposure to direct contact and/or handling by an operator of the catheter 101. The catheter cable 203 may be terminated at a PCBA 207 within the connector 209. The catheter cable 203 may be electrically and mechanically coupled to the imaging assembly 102 and may include a plurality of electrical wires.

In operation, a physician or a clinician may advance the catheter 101 into a lumen, such as a blood vessel, body lumen, or portion of a heart anatomy. By controlling actuators 116 and/or the clutch 114 on the handle 120, the physician or clinician may steer the catheter 101 to a position near the area of interest to be imaged. For example, one actuator may deflect the imaging assembly 102 and a distal end of the catheter cable 203 in a left-right plane and the other actuator may deflect the imaging assembly 102 and the distal end of the catheter cable 203 in an anterior-posterior plane. The clutch 114 may provide a locking mechanism to lock the positions of the actuators 116 and in effect lock the deflection of the imaging assembly 102 while imaging the area of interest.

The imaging process may include activating the ultrasound transducer elements on the imaging assembly 102 to produce ultrasonic energy. A portion of the ultrasonic energy is reflected by the area of interest and the surrounding anatomy, and the ultrasound echo signals are received by the ultrasound transducer elements. The handle 120 may be connected to the conduit 124 via another strain reliever. The conduit 124 may be configured to provide suitable configurations for interconnecting the control and processing system 130 and the monitor 132 to the imaging assembly 102. As such, the conduit 124 may be used to transfer the received echo signals to the control and processing system 130 where the ultrasound image is reconstructed and displayed on the monitor 132. In some embodiments, the processing system 130 can control the activation of the ultrasound transducer elements and the reception of the echo signals. In some embodiments, the control and processing system 130 and the monitor 132 may be part of a same system.

The system 100 and/or the ultrasound imaging device 110 may be utilized in a variety of applications such as transseptal punctures, left atrial appendage closures, atrial fibrillation ablation, and valve repairs and can be used to image vessels and structures within a living body. Although the system 100 is described in the context of intraluminal imaging procedures, the system 100 is suitable for use with any catheterization procedure. In addition, the imaging assembly 102 may include any suitable physiological sensor or component for diagnostic, treatment, and/or therapy. For example, the imaging assembly can include an imaging component, an ablation component, a cutting component, a morcellation component, a pressure-sensing component, a flow-sensing component, a temperature-sensing component, and/or combinations thereof. In some embodiments, the intraluminal imaging system 100 is used for generating two-dimensional and three-dimensional images.

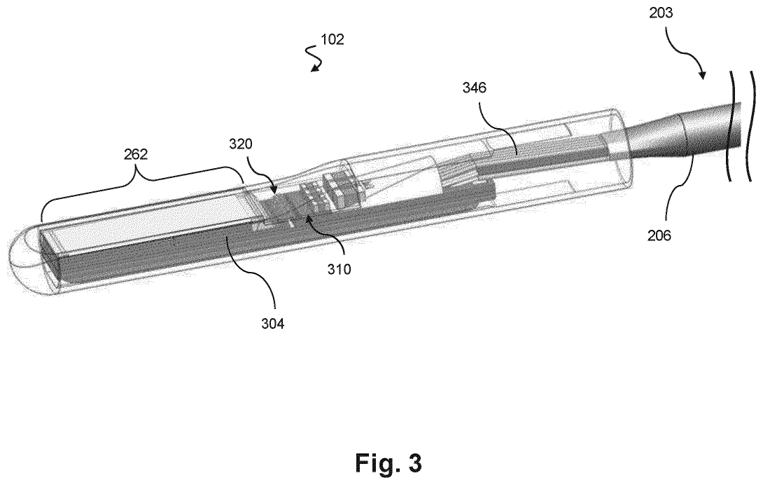

FIG. 3 is a perspective view of the imaging assembly 102 according to embodiments of the present disclosure. The imaging assembly 102 is positioned at the distal portion of the catheter shaft 201 after assembly. The imaging assembly 102 is also positioned at the distal portion of the cable 203. The imaging assembly 102 may include an ultrasound transducer array 262 that includes a number of transducer elements and a micro-beam-former IC 304 that can be coupled to the transducer array 262. The electrical wires 346 of the cable 203 are mechanically and/or electrical coupled to the imaging assembly 102. In some examples, the electrical cable 203 is further coupled through an interposer 310 to the micro-beam-former IC 304. In some examples the interposer 310 is connected to the micro-beam-former IC 304 through wire bonding 320. The wires 346 of the cable 203 are directly or indirectly in communication with the transducer array 262, the IC 304, and/or the interposer 310.

In some embodiments, the cable 203 includes a variety of electrical wires 346 or cables (e.g., lines and/or conductive pathways) configured to carry a variety of different electrical signals, such as data signals, power signals, control signals, and/or the like. For example, the cable 203 may include plurality of cables that allow communication of imaging data and/or command signals between the processing system 130 and the catheter 101. To that end, the cable 203 may extend between the imaging assembly 102 and the PCBA 207. Further, the cable 203 may include a number of signal lines (e.g., data signal lines) designated for transmitting the imaging data and/or additional data captured by the ultrasound imaging device 110 to the control and the processing system 130. The signal lines may further include a connection signal line. As described in greater detail below, the connection signal line may be configured to represent a state of connection between the ultrasound imaging device 110 and the control and processing system 130.

The cable 203 may also include control lines, which may carry control data from the control and processing system 130 to the ultrasound imaging device 110. In some instances, the control lines may include a serial databus, which may be used to program a component of the ultrasound imaging device 110, such as PCBA 207 and/or the micro-beam-former IC 304. Further, the cable 203 may include a number of power lines configured to provide power to one or more components of the ultrasound imaging device 110. For instance, the cable 203 may include a system ground power line and/or a ultrasound imaging device ground power line. In some cases, the system ground voltage may be the same as the ultrasound imaging device ground voltage. In such cases, a single ground power line may be used. In other embodiments, the ultrasound imaging device 110 may be configured to use a ground that floats relative to the system ground power line, so separate ground power lines may be used. The power lines may further include a high voltage (e.g., 65 volts (V)) power line, which may power the transducer array 262, and/or logic power lines which may provide a lower voltage (e.g., 1.8V, 3.3V, and/or the like) relative to the high voltage power line to other circuitry in the ultrasound imaging device 110, such as circuitry included in the PCBA 207, circuitry included in the micro-beam-former IC 304, and/or the like.

Moreover, while the signals carried on the electrical wires 346 have been described herein as data signals, control signals, and power signals, it may be appreciated that embodiments are not limited thereto and that any suitable signal may be carried on the electrical wires 346. For example, the electrical wires 346 may additionally or alternatively carry a clock signal (e.g., a digital clock), one or more system channel signals, and/or the like. Additionally, it may be appreciated that an electrical wire 346 may be configured for multiple uses. In this way, a particular signal may be transmitted on any suitable combination of electrical wires 346 and/or signal lines. Further, while the cable 203 is described herein as having electrical wire 346, the cable may additionally or alternatively include optical fibers, electrooptical fibers, and/or the like.

In some embodiments, the transducer array 262 includes ultrasound imaging transducers that are directly flip-chip mounted to the micro-beam-former IC 304. The transmitters and receivers of the ultrasound imaging transducers are on the micro-beam-former IC 304 and are directly attached to the transducers. In some examples, a mass termination of the acoustic elements is done at the micro-beam-former IC 304.

In some examples, the transducer array 262 includes more than 800 imaging elements and the electrical cable 203 includes a total of 12 signal lines or less. In some examples, the electrical cable 203 includes a total of 30 lines or less that includes the signal lines, power lines, and control lines, as described herein. In some examples, the transducer array 262 includes a one-dimensional or two-dimensional array from between 32 to 1000 imaging elements. For example, the array can include 32, 64, 128, 256, 512, 640, 768, or any other suitable number of imaging elements. For example, a one-dimensional array may have 32 imaging elements. A two-dimensional array may have 32, 64, or more imaging elements. In some examples, the number of signal lines is between 10 and 20, for example, 12 signal lines, 16 signal lines, or any other suitable number of signal lines. A one-dimensional array can be configured to generate two-dimensional images. A two-dimensional array can be configured to generate two-dimensional and/or three-dimensional images.

In some examples, the electrical cable 203 of the imaging assembly 102 is directly coupled to the micro-beam-former IC 304 of the imaging assembly 102. In some embodiments, the micro-beam-forming IC 304 lies directly underneath the transducer array 262 and is electrically connected to them. The elements of the transducer array 262 may be piezoelectric or micromachined ultrasonic transducer (MUT) elements. In some examples, piezoelectric elements are attached to the IC 304 by flip-chip mounting of an assembly of acoustic layers that include sawing into individual elements. MUT elements may be flip-chip mounted as a unit or grown directly on top of the micro-beam-forming IC 304. In some examples, the cable bundle may be terminated directly to the micro-beam-forming IC 304, or may be terminated to an interposer 310 of suitable material such as a rigid or flexible printed circuit assembly. The interposer 310 may then be connected to the micro-beam-forming IC 304 via any suitable means such as wire bondings 320.

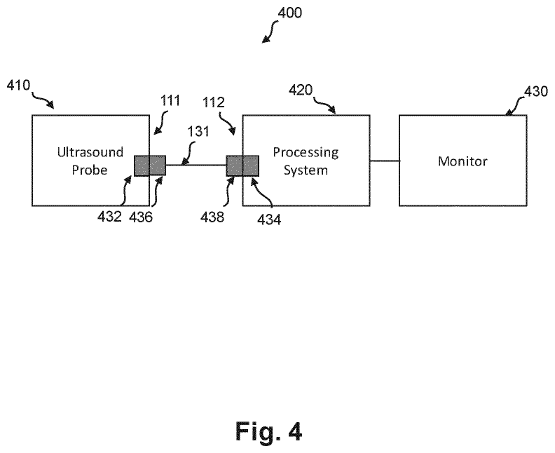

FIG. 4 is a block diagram of an imaging system 400 according to embodiments of the present disclosure. The system 400 may include an ultrasound probe 410 communicatively coupled to a processing system 420. The system may also include a monitor, such as display 430, communicatively coupled to the processing system 420, as illustrated.

The ultrasound probe 410 may be configured to capture ultrasound imaging data associated with a patient. Accordingly, the ultrasound probe 410 may include and/or be a component of the imaging device 110. In such embodiments, the ultrasound probe 410 may be included in an ICE catheter configured to capture intraluminal ultrasound imaging data. Additionally or alternatively, the ultrasound probe 410 may be included in an intravascular ultrasound (IVUS) device, an optical coherence tomography (OCT) device, an intracardiac echocardiography (ICE) device, a transesophageal echocardiography (TEE) device, an intravascular photoacoustic (IVPA) imaging device, and/or any suitable internal imaging device. Further, in some embodiments, the ultrasound probe 410 may be an external ultrasound imaging probe, such as a handheld ultrasound probe or a patch-based ultrasound probe. In such embodiments, the ultrasound probe 410 may be configured to capture ultrasound imaging data from a position external to the patient.

In some embodiments, the processing system 420 may include one or more computers, processors, and/or computer systems. For example, the processing system 420 may include one or more processors and/or memory modules forming a processing circuit that may process the electrical signals. As illustrated, the processing system 420 may be a stand-alone system (e.g., separate from the console 130). As such, the processing system 420 may output a graphical representation of the imaging data on the display 430. In other embodiments, the processing system 420 may be a component of the control and processing system 130.

In some embodiments, the ultrasound probe 410 may include a connector 432, such as a male connector (e.g., a plug), a female connector (e.g., a socket), or a hybrid connector having male and female connection components. The connector 432 may be located at any suitable location of the ultrasound probe. In some embodiments, for example, the connector 432 may correspond to a connection between a handle and a cable of the ultrasound probe 410 (e.g., between handle 120 and the conduit 124 and/or the cable 203). The connector 432 may be coupled to connector 436, such as the connector 209, via a cable, for example. The connector 432 and/or the connector 436 may facilitate electrical and mechanical connection (e.g., coupling) with the processing system 420. More specifically, the connector 432 and/or 436 may interface directly with the processing system 420 via one or more connectors (e.g., 434 and/or 438) of the processing system 420. In other embodiments, one of the connector 432 or the connector 436 may be omitted, and/or one of the connector 434 or 438 may be omitted. Additionally or alternatively, the connector 432 may interface with the processing system 420 via an indirect connection. In such cases, for example, the connector 436 may correspond to a distal connector of the PIM 131, and the connector 438 may correspond to a proximal connector of the PIM 131. In this way, the ultrasound probe 410 and the processing system 420 may be electrically coupled via one or more connector junctions, such as connector junction 111 and/or connector junction 112.

Each of the connectors (432, 434, 436, 438) may include one or more electrically conductive members (e.g., electrical pins, electrical pads, and/or the like) and/or optical members (e.g., optical fibers, optical connectors, electrooptical connectors, and/or the like) having any suitable shape, including cylindrical, planar surface(s), arcuate surface(s), or a combination thereof. The conductive members and/or optical members may facilitate electrical coupling and may enable communication between the ultrasound probe 410 and the processing system 420. In some embodiments, the conductive members may interface with the electrical wires 346 included in the cable 203 of the imaging device 110. Accordingly, the connectors (432, 434, 436, 438) may include conductive members respectively corresponding to a conductive pathway, such as a signal line, a power line, a control line, and/or the like. In this way, electrical signals representative of commands from the processing system 420 may be transmitted to the ultrasound probe 4100 via the connectors (432, 434, 436, 438). In some embodiments, proper electrical and/or physical connection between one or more of the connectors (432, 434, 436, 438) may reduce the transmission of improper electrical signals, such as electrical signals transmitted at an improper time or in an improper order, as described in greater detail below.

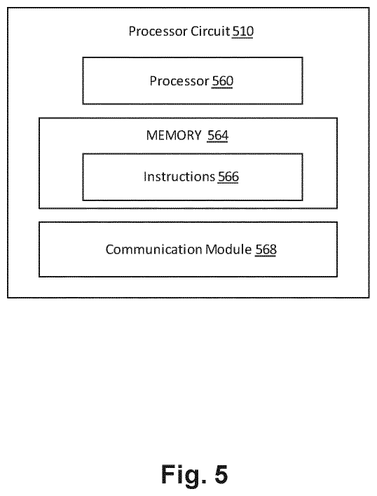

FIG. 5 is a schematic diagram of a processor circuit 510, according to aspects of the present disclosure. The processor circuit 510 or a similar processor circuit may be implemented in any suitable device or system previously disclosed. One or more processor circuits 510 can be configured to perform the operations described herein. The processor circuit 510 can include additional circuitry or electronic components, such as those described herein. In an example, the control and processing system 130 includes one or more processor circuits 510. In some embodiments, one or more processor circuits 510 may be in communication with transducer arrays, sensors, circuitry, or other components within the ultrasound imaging device 110 and/or the control and processing system 130. One or more processor circuits 510 may also be in communication with the monitor 132, as well as any other suitable component or circuit within the imaging system 100. As shown, the processor circuit 510 may include a processor 560, a memory 564, and a communication module 568. These elements may be in direct or indirect communication with each other, for example via one or more buses.

The processor 560 may include a CPU, a GPU, a DSP, an application-specific integrated circuit (ASIC), a controller, a field programmable gate array (FPGA), another hardware device, a firmware device, or any combination thereof configured to perform the operations described herein. The processor 560 may also be implemented as a combination of computing devices, e.g., a combination of a DSP and a microprocessor, a plurality of microprocessors, one or more microprocessors in conjunction with a DSP core, or any other such configuration.

The memory 564 may include a cache memory (e.g., a cache memory of the processor 560), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an embodiment, the memory 564 includes a non-transitory computer-readable medium. The memory 564 may store instructions 566. The instructions 566 may include instructions that, when executed by the processor 560, cause the processor 560 to perform the operations described herein with reference to the device 110, and/or the system 130. Instructions 566 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 568 can include any electronic circuitry and/or logic circuitry to facilitate direct or indirect communication of data between the processor circuit 510, the previously described devices and systems, and/or the monitor 132. In that regard, the communication module 568 can be an input/output (I/O) device. In some instances, the communication module 568 facilitates direct or indirect communication between various elements of the processor circuit 510 and/or the devices and systems of the imaging system 100.

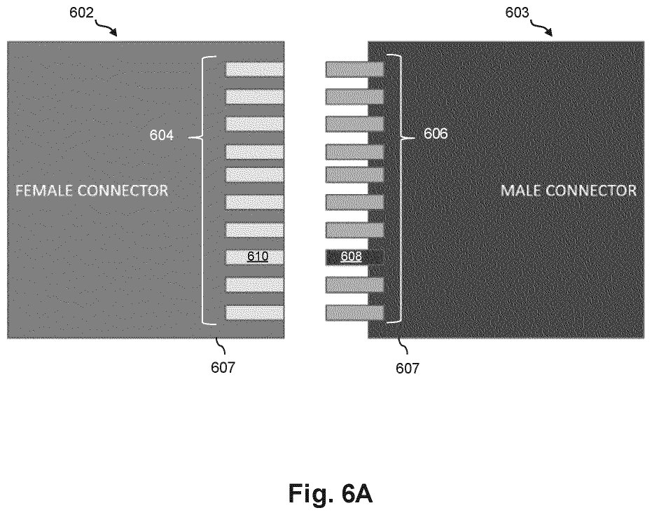

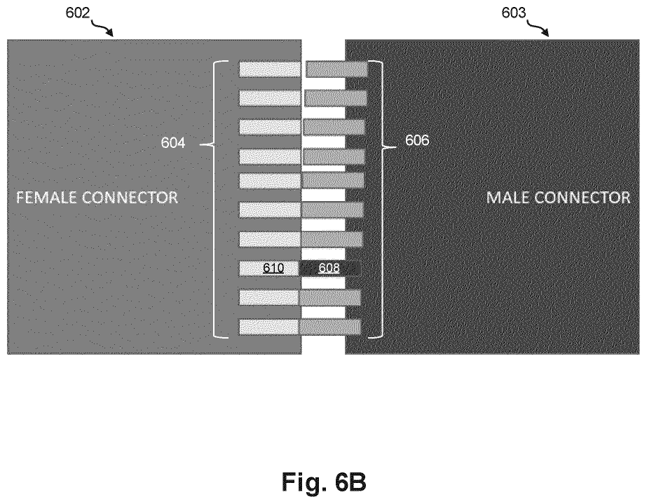

Turning now to FIGS. 6A-B, a schematic diagram of a female connector 602 and a male connector 603 is illustrated. The female connector 602 and the male connector 603 may represent any two connectors (e.g., 432, 434, 436, 438) at a connector junction (e.g., 111, 112), as described herein. To that end, the female connector 602 and/or the male connector 603 may be included in the ultrasound probe 410, the processing system 420, a cable connecting the ultrasound probe 410 and the processing system 420, such as the PIM 131, and/or the like. However, for the purposes of example, the female connector 602 is described herein as being incorporated in the ultrasound probe 410, and the male connector 603 is described herein as being incorporated in the processing system 420. Additionally, while embodiments described herein reference coupling between a female connector and a male connector, it may be appreciated that embodiments are not limited thereto and that hybrid connectors having both male and female components may be used.

As illustrated, the female connector 602 includes a set of electrical pads 604, which may be configured to receive and electrically couple to a set of electrical pins 606 on the male connector 603. Each of the electrical pads 604 and the electrical pins 606 may be dedicated to signal lines, control lines, power lines, and/or the like that correspond to the electrical wires 346 in the ultrasound probe 410 described above. In some embodiments, the electrical pads 604 may be electrically coupled to the electrical wires 346 in the cable 203 via a direct and/or fixed connection (e.g., via soldering). As the electrical pins 606 are configured to electrically couple to the electrical pads 604, the electrical pads 604 may be dedicated to the same combination of signal lines, control lines, power lines and/or the like as the electrical pins 606. As further illustrated, the electrical pins 606 and the electrical pads 604 may be positioned on or within a body 607 (e.g., a housing) of a connector (603 and 602, respectively).

Additionally, the male connector 603 may include a connection pin 608 (e.g., one or more conductive members having any suitable shape, including cylindrical, planar surface(s), arcuate surface(s), and/or combinations thereof) configured to electrically couple to a connection pad 610 (e.g., one or more conductive members having any suitable shape, including cylindrical, planar surface(s), arcuate surface(s), and/or combinations thereof) of the female connector 602. In some cases, an electrical pin 606 may be designated as the connection pin 608 on the male connector 603. Similarly, an electrical pad 604 may be designated as the connection pad 610 on the female connector 602. The connection pin 608 and the connection pad 610 may electrically couple to a signal line configured to indicate a status of connection between two devices (e.g., a connection signal line), such as the ultrasound probe 410 and the processing system 420. In some embodiments, for example, an electrical signal transmitted on the connection signal line via the connection pin 608 and the connection pad 610 may transition from a first state to a second state (e.g., from a low state to a high state, a high state to a low state, and/or the like) to indicate that the ultrasound probe 410 and the processing system 420 are electrically connected. To that end, the electrical conductance on the connection signal line may transition from a first state to a second state. Accordingly, in some embodiments, the processing system 420 may determine that the ultrasound probe 410 is electrically connected to the processing system 420 based on the connection signal line. In this way, a signal and/or conductance transmitted via the connection pin 608 and the connection pad 610 may indicate that the ultrasound probe 410 and/or the processing system 420 may begin transmission of control and/or communication data over the signal lines, control lines, power lines, and/or the like included in the connectors (602, 603).

As illustrated in FIG. 6B, in some cases, an electrical connection may be formed between the connection pin 608 and the connection pad 610 before an electrical connection is formed at each of the electrical pins 606 and the corresponding electrical pads 604. As such, a signal indicating that a connection is formed between the ultrasound probe 410 and the processing system 420 and/or that communication between the ultrasound probe 410 and the processing system 420 may be transmitted before each of the electrical pins 606 corresponding to power lines are electrically coupled to the corresponding electrical pads 604. Moreover, in some instances, the processing system 420 may be powered on (e.g., hot) while coupling to the ultrasound probe 410. Thus, in some instances, the processing system 420 may be able to transmit communication data and/or control signals to the ultrasound probe 410 before power is provided to one or more components of the ultrasound probe 410. For example, as described above, the communication data and/or control signals may be transmitted before power is provided to the transducer array 262, the micro-beam-former IC 304, the PCBA 207, and/or the like, which may be configured to be powered by one or more voltages delivered over power lines within the cable 203. As a result, information transmitted to an unpowered component in the ultrasound probe 410 may be lost and/or may not be received, which may cause the ultrasound probe 410 to malfunction and/or overheat.

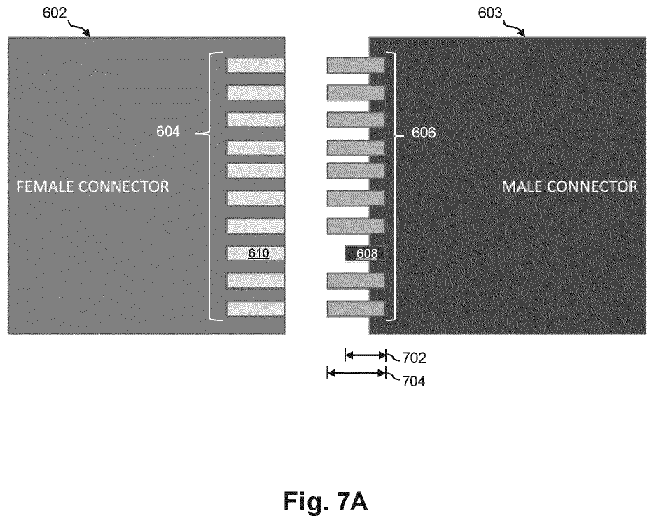

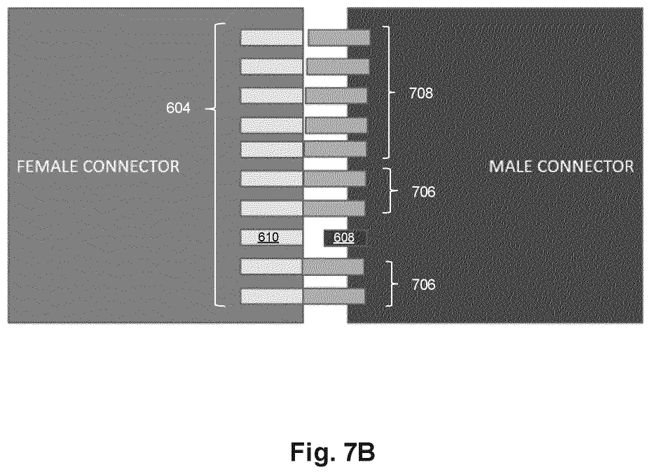

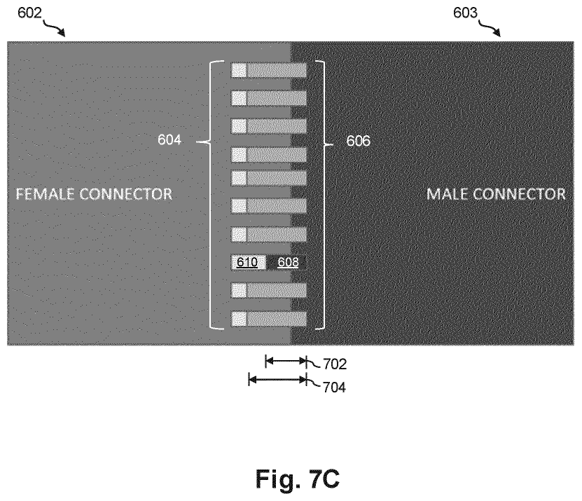

Accordingly, one or more connectors used at a connection junction (e.g., 111, 112) between the ultrasound probe 410 and the processing system 420 may be modified to prevent data transmission to unpowered components of the ultrasound probe 410, which may reduce malfunctions in the ultrasound probe 410. More specifically, in some embodiments, the connectors, such as female connector 602 and/or male connector 603, may be modified so that the connection pin 608 forms an electrical connection with the connection pad 610 after the electrical pins 606 corresponding to power lines form an electrical connection with the corresponding electrical pads 604. Thus, as described with reference to FIGS. 7-15, the length and/or position of one or more electrical pins 606 and/or electrical pads 604, such as the connection pin 608 and/or the connection pad 610, may be adjusted within the connectors (602, 603). Additionally or alternatively, the female connector 602 and/or the male connector 603 may include an impedance element configured to delay a rise-time on the connection signal path. Moreover, in some embodiments, the processing system 420 may determine that the ultrasound probe 410 is connected based on the connection signal corresponding to the connection pin, as well as an additional connection signal corresponding to an additional connection pin, as described below. In this way, the connection pin 608 and/or the connection pad 610 may be configured so that electrical coupling between the connection pin 608 and the connection pad 610 indicates full electrical coupling and/or mechanical coupling between the female connector 602 and the male connector 603.

FIGS. 7A-C are schematic diagrams illustrating coupling of a female connector 602 with a male connector 603 that includes a shortened connection pin 608. That is, the connection pin 608 is shorter than the other electrical pins 606 of the male connector 603. As such, a length 702 of the connection pin 608 is less than a length 704 of the other electrical pins 606. In some embodiments, the difference between the length 702 and the length 704 may be selected such that the connection pin 608 is the last pin to form a connection with an electrical pad 604 (e.g., connection pad 610). In other words, during coupling (e.g., electrical and mechanical coupling) of the female connector 602 and the male connector 603, the connection pin 608 is the last pin to form a connection with an electrical pad 604, regardless of the orientation of the female connector 602 or the male connector 603. Accordingly, in some embodiments, the processing system 420 may be triggered to monitor the connection signal line corresponding to the connection pin 608 to determine the connection stats of the connection pin 608 after an electrical pin 606 forms an electrical connection with an electrical pad 604. Moreover, by initiating transmission of data (e.g., control data and/or communication data) based on the connection status of the connection pin 608, the transmission is ensured to begin after power is delivered to each of the components of the ultrasound probe 410 (e.g., after each of the electrical pins 606 coupled to a power line electrically couple to the corresponding electrical pads 604).

For example, FIG. 7B illustrates the male connector 603 being brought into contact with the female connector 602 for electrical and mechanical coupling. As illustrated, while a first set of electrical pins 706 on the male connector 603 are in contact and may form an electrical connection with a corresponding set of electrical pads 604 on the female connector 602, a second set of electrical pins 708, as well as the connection pin 608, remain disconnected (e.g., physically and electrically separated) from the female connector 602. Because the connection pin 608 remains disconnected, transmission of data between the ultrasound probe 410 and the processing system 420 may remain uninitiated. Thus, even if any of first set of electrical pins 708 are coupled to a control line or a signal line and the processing system 420 is powered, the processing system 420 may be prevented from transmitting data when the connectors (602, 603) are in the illustrated state. Accordingly, each of the second set of electrical pins 708, which may include electrical pins 606 coupled to a power line, may form an electrical connection with the corresponding electrical pads 604 prior to initiation of data transmission between the ultrasound probe 410 and the processing system 420.

FIG. 7C illustrates a full mechanical and electrical connection between the female connector 602 and the male connector 603. To that end, for example, each of the electrical pins 606 is in electrical connection with a corresponding electrical pad 604. As a result, a conductive pathway may be formed between each of the electrical pins 606 and the corresponding electrical pads 604. As described herein, the conductive pathways may correspond to the lines (e.g., electrical wires 346) within the cable 203, such as power lines, control lines, signal lines, and/or the like. As further illustrated, the length 702 may be selected such that the connection pin 608 is able to form an electrical connection and mechanically engage with the connection pad 610. Further, each of the electrical pins 606 may physically engage with the corresponding electrical pads 604 to form a secure mechanical connection. Additionally or alternatively, the female connector 602 and/or the male connector 603 may include a feature, such as a snap-fit feature, a locking mechanism, and/or the like to establish the mechanical connection between the female connector 602 and the male connector 603.

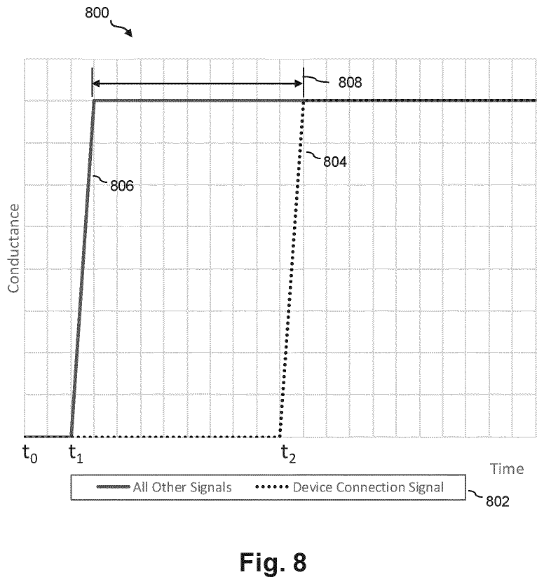

Turning now to FIG. 8, a timing diagram 800 of the electrical coupling between the electrical pins 606 of the male connector 603 and the electrical pads 604 of the female connector 602 of FIGS. 7A-C is illustrated. As indicated in the legend 802, the timing diagram 800 includes a first curve 804 corresponding to a device connection signal line and a second curve 806 corresponding to other conductive pathways, such as power lines, control lines, data signal lines, and/or the like. More specifically, the first curve 804 corresponds to the conductance (e.g., in siemens) measured on the connection signal line over time (e.g., in milliseconds). For example, the first curve 804 may correspond to the conductance measured at the connection pin 608, the connection pad 610, or any other suitable location along the connection signal line conductive path. Similarly, the second curve 806 may correspond to the conductance measured on the lines coupled to the other electrical pins 606 (e.g., excluding the connection pin 608) over time.

For the purposes of example, a single curve (806, 1006, 1206, and 1406) is illustrated for the other electrical pins 606 in FIGS. 8, 10, 12, and 14, respectively. However, it may be appreciated that the conductance of each of lines coupled to the electrical pins 606 may vary and may be measured individually. Further, while the timing diagrams 800, 1000, 1200, and 1400 of FIGS. 8, 10, 12, and 14, respectively are described herein in terms of conductance, it may be appreciated that any suitable measurement, such as a voltage, current, resistance, and/or the like, on the connection signal lines and the other lines may be performed and that embodiments are not limited thereto.

As illustrated in the timing diagram 800, at an initial time (t0), the electrical pins 606 are not electrically coupled to the electrical pads 604, as illustrated in FIG. 7A. Accordingly, a conductance may not be present on the conductive pathways corresponding to the electrical pins 606 and/or the electrical pads 604. At a first time (t1), an electrical pin 606 may begin to electrically couple to a corresponding electrical pad 604, as illustrated by the first set of electrical pins 706 in FIG. 7B. Accordingly, a conductance may be measured on the conductive pathway corresponding to the electrical pin 606, and, as illustrated, the second curve 806 rises at the first time (t1) until a full electrical coupling is established. Similarly, at a second time (t2), the connection pin 608 may begin to electrically couple to the connection pad 610, as illustrated in FIG. 7C. Thus, a conductance may be measured on the conductive pathway corresponding to the connection pin 608, and, as illustrated, the first curve 804 rises at the second time (t2) until a full electrical coupling is established. In some embodiments, the processing system 420 may be configured to monitor the conductive pathway corresponding to the connection pin (e.g., monitor the second curve 806) in response to determining that the electrical pin 606 has formed an electrical connection with the electrical pad 604 (e.g., that the first curve 804 has risen). In other embodiments, the processing system 420 may monitor the conductive pathway on a regular, periodic interval or may be configured to interrupt when a change in conductance occurs on the conductive pathway.

In some embodiments, a duration 808 between the full electrical coupling of the electrical pin 606 with the electrical pad 604 and the full electrical coupling of the connection pin 608 with the connection electrical pad 610 may correspond to a time taken to mechanically actuate the male connector 603 or the female connector 602 from a first position where the electrical pin 606 is electrically coupled to the electrical pad 604 and the connection pin 608 is not electrically coupled, as illustrated in FIG. 7B, to a second position where the connection pin 608 is electrically coupled to the connection pad 610, as illustrated in FIG. 7C. In this way, the duration 808 may correspond to the difference in length between the length 702 and the length 704, as well as the speed at which the female connector 602 and the male connector 603 are coupled together. Moreover, the duration 808 may correspond to a minimum time elapsed before data transmission between the ultrasound probe 410 and the processing system 420 is initiated. For example, processing system 420 may be configured to detect the full electrical coupling between the connection pin 608 and the connection pad 610 based on the conductance of the connection signal path (e.g., the first curve 804) and configured to initiate data transmission only after detecting this coupling.

Further, in some embodiments, the processing system 420 and/or the ultrasound probe 410 may be configured to identify a connection error based on the duration 808. For example, in response to determining that the duration 808 (e.g., a time elapsed since the electrical pin 606 establishes an electrical connection with an electrical pad 604) exceeds a certain threshold, the processing system 420 may determine that an improper and/or incomplete coupling between the male connector 603 and the female connector 602 has occurred. In some instances, for example, the male connector 603 may not be fully inserted into the female connector 602, which may prevent the connection pin 608 from coupling with the connection pad 610. In response to determining that an improper or an incomplete coupling between connectors has occurred, the processing system 420 may output a visual alert and/or message to the display 430 for display and/or output an audible alert and/or message to a speaker. The alert(s) and/or message(s) may advise a user to reconnect the connectors.

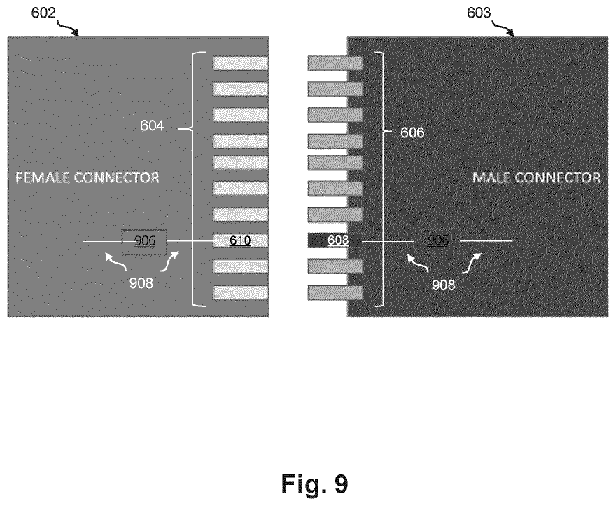

FIG. 9 illustrates a schematic diagram of an impedance element 906 integrated in the female connector 602 and the male connector 603. The impedance element 906 may be selected to slow or delay a rise time of a connection signal on the connection signal line conductive pathway. Thus, as illustrated, the impedance element 906 may be coupled to the connection pin 608, the connection pad 610, or both. The impedance element 906 may be coupled to the connection pin 608 and/or pad 610 via a direct connection (e.g., via one or more wires 908) or an indirect connection. In some embodiments, the impedance element 906 may be a passive impedance element, such as a resistor, an inductor, a capacitor, or a combination thereof. Moreover, the impedance element 906 may be positioned to affect the rise time of the connection signal at any suitable location in the connection signal line conductive pathway (e.g., within or external to a connector). As such, the impedance element 906 may be included in any suitable location of the ultrasound probe 410, the processing system 420, or both. By including the impedance element in the processing system 420, such as in a connector of the processing system (e.g., 434 and/or 438), the rise time on the connection signal line conductive pathway may be delayed regardless of the type of probe or device connected to the processing system 420. On the other hand, by including the impedance element 906 in only the ultrasound probe 410, the rise time on the connection signal line conductive pathway may be delayed for the ultrasound probe 410 and may remain unchanged for a different probe or device connected to the processing system 420. In this way, probe-specific rise-time delays may be implemented using an impedance element 906 in the ultrasound probe 410.

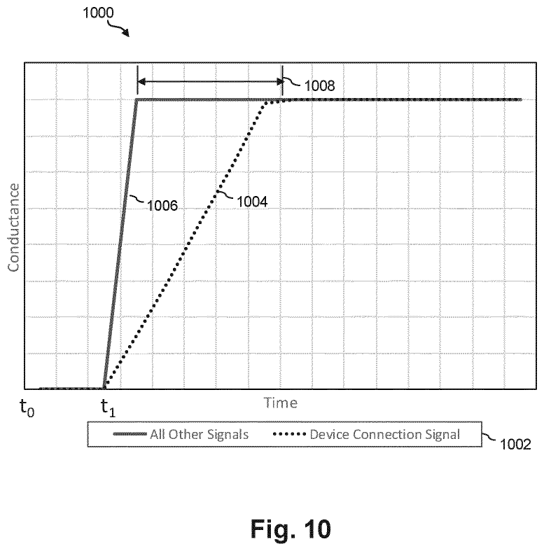

FIG. 10 illustrates a timing diagram 1000 of the electrical coupling between the electrical pins 606 of the male connector 603 and the electrical pads 604 of the female connector 602 of FIG. 9. As indicated in the legend 1002, the timing diagram 1000 includes a first curve 1004 corresponding to a device connection signal line and a second curve 1006 corresponding to other conductive pathways, such as power lines, control lines, data signal lines, and/or the like.

As illustrated, each of the electrical pins 606 of the male connector 603, including the connection pin 608, may begin to electrically couple to the corresponding electrical pads 604, including the connection pad 610, resulting in a change in conductance at time t1. For example, at t1, each of the electrical pins 606 may physically and electrically contact (e.g., engage) the corresponding electrical pads 604, and after a conductance on the conductive pathway corresponding to the electrical pin 606 rises to a certain level, a full electrical connection between the electrical pin 606 and the electrical pad 604 may be formed. The time between the initial electrical contact and the conductance rising to the level of a full electrical connection may be referred to as rise-time. Moreover, as illustrated by the timing diagram 1000, the rise-time of the conductive pathway corresponding to the connection pin 608 may be slowed or delayed relative to the rise-time of another conductive pathway (e.g., corresponding to the second curve 1006) by a duration 1008. The duration 1008 may result from the impedance element 906, as described above with reference to FIG. 9. Accordingly, in some embodiments the duration 1008 may be adjusted based on selection of the impedance element 906. Further, because initialization of data communication between the processing system 420 and the ultrasound probe 410 may depend on the full electrical connection between the connection pin 608 and the connection pad 610, the duration 1008 may correspond to a minimum time elapsed before data transmission between the ultrasound probe 410 and the processing system 420 is initiated, as described herein.

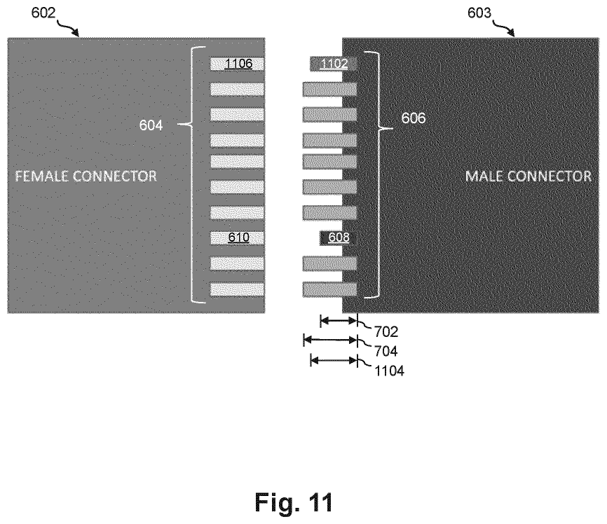

Turning to FIG. 11, a schematic diagram of the female connector 602 and a male connector 603 having a connection pin 608 and an additional connection pin 1102 is illustrated. As described above with reference to the connection pin 606, the additional connection pin 1102 may couple to an additional connection pad 1106. Both the additional connection pin 1102 and the additional connection pad 1106 may be coupled to an additional connection signal line, which may be configured to indicate a status of connection between the female connector 602 and the male connector 603. In this way, the status of connection between the female connector 602 and the male connector 603 may be determined based on the connection signal line and the additional connection signal line. Thus, in some embodiments, initialization of data transmission may be based on both coupling of the additional connection pin 1102 and coupling of the connection pin 608. For example, the processing system 420 may be configured to initialize data communication with the ultrasound probe 410 only after detecting a conductance on both the additional connection signal line and the connection signal line.

In some embodiments, a length 1104 of the additional connection pin 1102 may be offset from both the other electrical pins 606 and the connection pin 608. For example, in the illustrated embodiment, the length 704 of the electrical pins 606 is greater than the length 1104 of the additional connection pin 1102, which, in turn, is greater than the length 702 of the connection pin 608. Accordingly, the additional connection pin 1102 may couple to the additional connection pad 1106 after an electrical pin 606 couples to an electrical pad 604, and the connection pin 608 may electrically couple to the connection pad 610 after the additional connection pin 1102 couples to the additional connection pad 1106. Thus, in some embodiments, connection of the additional connection pin 1102 with the additional connection pad 1106 may be used as a reference to predict when connection of the connection pin 608 with the connection pad 610 will occur. In this way, the processing system 420 may control initialization of data transmission based on the timing of the coupling of the connection pin 608 with reference to the timing of the additional connection pin 1102, as described below.

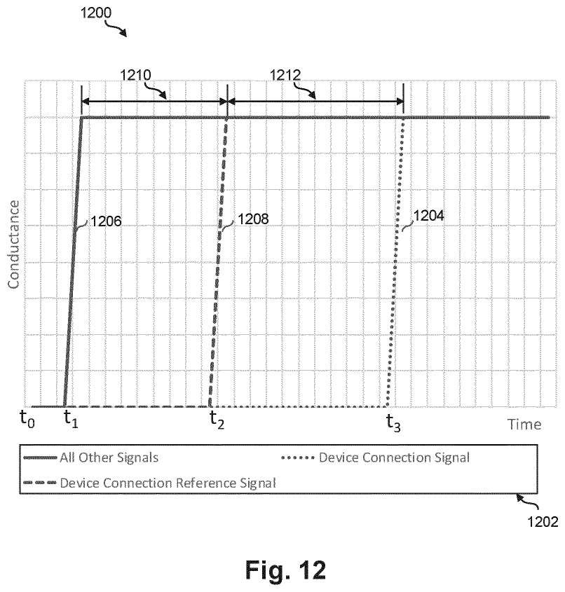

FIG. 12 illustrates a timing diagram 1200 of the electrical coupling between the electrical pins 606 of the male connector 603 and the electrical pads 604 of the female connector 602 of FIG. 11. As indicated in the legend 1202, the timing diagram 1200 includes a first curve 1204 corresponding to a device connection signal, a second curve 1206 corresponding to other signals, such as power signals, control signals, data signals, and/or the like, and a third curve 1208 corresponding to a device connection reference signal. As described above with reference to FIG. 8, the first curve 1204 corresponds to the conductance (e.g., in siemens) measured on the connection signal line over time (e.g., in milliseconds), and the second curve 1206 may correspond to the conductance measured on the lines coupled to the other electrical pins 606 (e.g., excluding the connection pin 608) over time. Further, the third curve 1208 may correspond to the conductance over time measured on an additional connection signal line, which may be coupled to the additional connection pin 1102.

As illustrated, each of the electrical pins 606 having a length 704 may begin to electrically couple to the corresponding electrical pads 604 at a first time (t1). For example, because the electrical pins 606 having the length 704 are the longest pins on the male connector 603, they may physically contact the electrical pads 604 before (e.g., at the first time (t1)) either the additional connection pin 1102 or the connection pin 608 contact the additional connection pad 1106 or the connection pad 610, respectively. Subsequently, at a second time (t2), the additional connection pin 1102 may electrically contact the additional connection pad 1106, and a full electrical connection between the additional connection pin 1102 and the additional connection pad 1106 may be formed. At a third time (t3), the connection pin 608 may electrically contact the connection pad 610, and a full electrical connection between the connection pin 608 and the connection pad 610 may be formed. Further, because the length 702 of the connection pin 608 is less than both the length 704 and the length 1104, the connection pin 608 and the connection pad 610 may form the final electrical connection between the male connector 603 and the female connector 602, completing electrical coupling between the ultrasound probe 410 and the processing system 420.

In some embodiments, a duration 1210 between the formation of the electrical connection between an electrical pin 606 having a length 704 and an electrical pad 604 and the formation of the electrical connection between the additional connection pin 1102 and the additional connection pad 1106 may be determined. The duration 1210 may depend on a difference between the length 704 and the length 1104, as well as a speed the male connector 603 and the female connector 602 are brought into contact. In this way, the duration 1210 may be reduced if the speed at which the male connector 603 and the female connector 602 are brought into contact increases and/or the difference between the length 704 and the length 1104 is reduced. Further, a duration 1212 between the formation of the electrical connection between the additional connection pin 1102 and the additional connection pad 1106 and formation of the electrical connection between the connection pin 608 and the connection pad 610 may be determined. The duration 1212 may depend on a difference between the length 702 and the length 1104, as well as a speed the male connector 603 and the female connector 602 are brought into contact.

The duration 1210 and the duration 1212 may be used to identify connection issues, such as improper or incomplete connection between the male connector 603 and the female connector 602. For example, in some embodiments, the duration 1210 may be used to predict the duration 1212. For instance, based on the duration 1210, the length 704, and the length 1104, a speed at which the male connector 603 and the female connector 602 may be determined. Using the speed, the length 702, and the length 1104, a time for the electrical connection between the connection pin 608 and the connection pad 610 to form may be estimated. Additionally or alternatively, the time for electrical connection between the connection pin 608 and the connection pad 610 may be estimated based on a relationship (e.g., a ratio) between the length 702, the length 704, the length 1104, or a combination thereof and the duration 1210. In any case, estimated time may then be compared to the actual duration 1212. In some embodiments, a difference between the estimated time and the duration 1212 exceeding a certain threshold (e.g., 1 ms, 5 ms, 10 ms, and/or the like) may indicate that a connection error has occurred. In such cases, the processing system 420 may be configured to output a visual alert and/or message to the display 430 for display and/or output an audible alert and/or message to a speaker. The alert(s) and/or message(s) may advise a user to reconnect the connectors. Further, in some embodiments, data communication between the processing system 420 and the ultrasound probe 410 may be prevented until conductance is detected on both a conductive pathway corresponding to the additional connection pin 1102 and a conductive pathway corresponding to the connection pin 608. Accordingly, a connection issue may be detected based on the duration 1210 exceeding a threshold, the duration 1212 exceeding a threshold, or both.

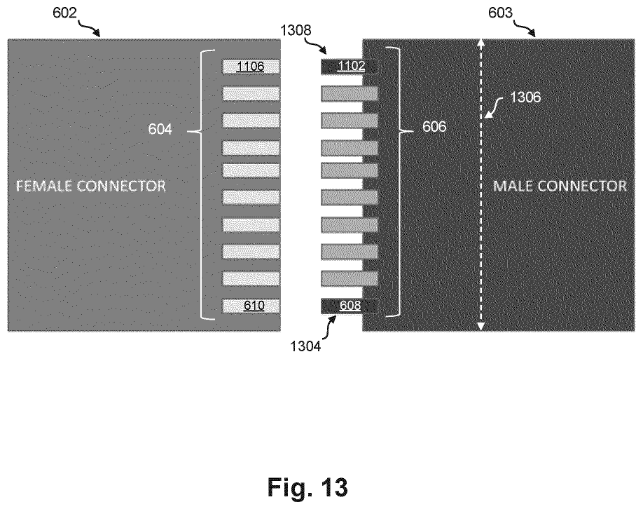

FIG. 13 is a schematic diagram of the female connector 602 and a male connector 603 having a connection pin 608 and an additional connection pin 1102 spaced from one another along a longitudinal axis 1306. As illustrated, the connection pin 608 may be positioned as a first end pin 1304 of the male connector 603 such that other electrical pins 606 are positioned adjacent to the connection pin 608 only in a single, first direction along the longitudinal axis 1306. Similarly, the additional connection pin 1102 may be positioned as a second end pin 1308 such that other electrical pins 606 are positioned adjacent to the additional connection pin 1102 only in a single, second direction along the longitudinal axis 1306 opposite the first direction. By spacing the connection pin 608 and the additional connection pin 1102 at opposite ends (e.g., 1304 and 1308, respectively), the connection pin 608 or the additional connection pin 1102 may contact an electrical pad (e.g., the connection pad 610 or an additional connection pad 1106, respectively) before other electrical pins 606 of the male connector 603. To that end, regardless of an angle of coupling between the female connector 602 and the male connector 603, at least one conductive pathway corresponding to a connection signal line may be formed before any conductive pathway corresponding to a signal line, a control line, or a power line is formed.

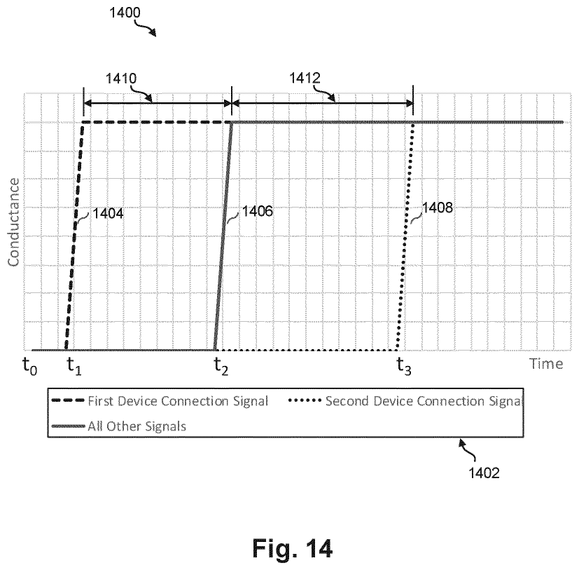

FIG. 14 illustrates a timing diagram 1400 of the electrical coupling between the electrical pins 606 of the male connector 603 and the electrical pads 604 of the female connector 602 of FIG. 13. As indicated in the legend 1402, the timing diagram 1400 includes a first curve 1404 corresponding to a first device connection signal, a second curve 1406 corresponding to other conductive pathways, such as power signal lines, control signal lines, data signal lines, and/or the like, and a third curve 1408 corresponding to a second device connection signal. The first curve 1404 may correspond to a conductive pathway coupled to the connection pin 608, and the third curve 1408 may correspond to a conductive pathway coupled to the additional connection pin 1102 or vice versa.

Further, the timing diagram 1400 may correspond to connection of the male connector 603 of FIG. 13 angled relative to the female connector 602 of FIG. 13. Accordingly, the electrical coupling between electrical pins 606, including the connection pin 608 and the additional connection pin 1102, and the electrical pads 604 may be distributed across time. Thus, as illustrated, the connection pin 608 may begin to electrically couple to the connection pad 610 at a first time (t1). For example, if the end pin 1304 is tilted toward the female connector 602 and the end pin 1308 is tilted away from the female connector 602, the connection pin 608 may form the first electrical connection with an electrical pad (e.g., the connection pad 610) of the female connector 602. Subsequently, at a second time (t2), the electrical pins 606 may contact the electrical pads 604, and a full electrical connection between the electrical pins 606 and the electrical pads 604 may be formed. At a third time (t3), the additional connection pin 1102, which may have been tilted away from the female connector 602 during connection, may electrically contact the additional connection pad 1106. A full electrical connection between the additional connection pin 1102 and the additional connection pad 1106 may then be formed. Thus, by initiating data communication between the ultrasound probe 410 and the processing system 420 only after each of the connection pin 606 and the additional connection pin 1102 form full electrical connections with the female connector 602, the data communication may be initiated after each electrical pin 606 corresponding to a power line is electrically connected to the female connector 602. Further, while the illustrated timing diagram 1400 corresponds to connection between the male connector 603 and the female connector 602 at an angle, each of the electrical pins 606, including the connection pin 606 and the additional connection pin 1102, may form an electrical connection at the female connector 602 at substantially the same time when the male connector and the female connector 602 are connected in complete alignment. Accordingly, using the connection pin 606 and the additional connection pin 1102 for reference, data communication may still be initiated after each electrical pin 606 corresponding to a power line is electrically connected to the female connector 602.

In some embodiments, a duration 1410 between the formation of the electrical connection between the connection pin 608 and the connection pad 610 and the formation of the electrical connection between an electrical pin 606 an electrical pad 604 may be determined. Similarly, a duration 1412 between the formation of the electrical connection between an electrical pin 606 an electrical pad 604 and the formation of the electrical connection between the additional connection pin 1102 and the additional connection pad 1106 may be determined. The duration 1410, the duration 1412, and or a total duration, which may include a sum of the duration 1410 and the duration 1412, may be used to identify connection issues, as described herein.

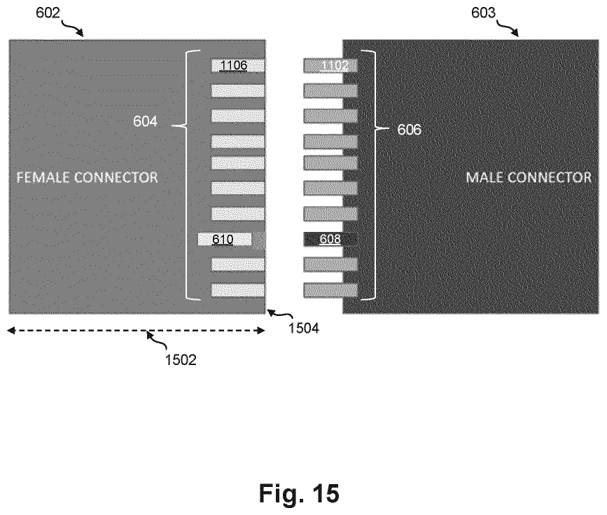

FIG. 15 a is schematic diagram of the male connector 603 and a female connector 602 having a connection pad 610 offset along a lateral axis 1502 from an edge 1504 of the female connector 602. As illustrated, the connection pad 610 is positioned further, along the lateral axis 1502, from the edge 1504 of the female connector 602 than the other electrical pads 604. Moreover, in the illustrated embodiment, each of the electrical pins 606, including the connection pin 608, have an equal length (e.g., length 704). As such, the connection pad 610 may be positioned such that the connection pin 608 is the last electrical pin 606 to form a connection with a corresponding electrical pad 604. Thus, similar to the coupling (e.g., electrical and mechanical coupling) of the female connector 602 and the male connector 603 described with reference to FIGS. 7A-C, the connection pin 608 is the last pin to form a connection at the female connector 602 (e.g., a connection with the connection pad 610), regardless of the orientation of the female connector 602 or the male connector 603. Accordingly, by initiating transmission of data (e.g., control data and/or communication data) based on the connection status of the connection pin 608, the transmission is ensured to begin after power is delivered to each of the components of the ultrasound probe 410 (e.g., after each of the electrical pins 606 coupled to a power line is electrically coupled to the corresponding electrical pads 604).

While the illustrated embodiment depicts the position of the connection pad 610 as being offset relative to the edge 1504 along the lateral axis 1502, the position of connection pad 610 may additionally or alternatively be offset relative to the other electrical pads 604 along the lateral axis 1502. Further, in some embodiments, each of the electrical pads 604, including the connection pad 610, may be offset relative to the edge 1504 along the axis 1502 such that the each of the electrical pins 606 are mechanically guided into alignment such that the electrical pins 606 relatively simultaneously contact the corresponding electrical pads 604.

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. An ultrasound imaging system, comprising:
an ultrasound probe comprising an ultrasound transducer array;
a processor circuit configured for communication with the ultrasound probe via a first conductive pathway and a second conductive pathway; and
a first connector and a second connector configured to be selectively engaged to establish the communication between the ultrasound probe and the processor circuit,
wherein the processor circuit is configured to:
detect an electrical conductance along the first conductive pathway; and
transmit data to the ultrasound probe via the second conductive pathway only after detecting the electrical conductance along the first conductive pathway.

2. The system of claim 1,
wherein the first conductive pathway comprises a first conductive member of the first connector and a first conductive member of the second connector, and
wherein the second conductive pathway comprises a second conductive member of the first connector and a second conductive member of the second connector.

3. The system of claim 2, wherein a first length of the first conductive member of the first connector and a second length of the second conductive member of the first connector are different.

4. The system of claim 3, wherein the first length is less than the second length.

5. The system of claim 2, wherein the first conductive member of the first connector is offset along a lateral axis of the first connector relative to the second conductive member of the first connector.

6. The system of claim 2, wherein the first conductive pathway comprises an impedance element configured to delay the processor circuit detecting the electrical conductance along the first conductive pathway.

7. The system of claim 6, wherein the first connector comprises the impedance element.

8. The system of claim 6, wherein the second connector comprises the impedance element.

9. The system of claim 2,
wherein the processor circuit is further configured for communication with the ultrasound probe via a third conductive pathway,
wherein the third conductive pathway comprises a third conductive member of the first connector and a third conductive member of the second connector,
wherein the first conductive member of the first connector is disposed at a first end of the first connector,
wherein the third conductive member of the first connector is disposed at an opposite, second end of the first connector,
wherein the first conductive member of the second connector is disposed at a first end of the second connector, and
wherein the third conductive member of the second connector is disposed at an opposite, second end of the second connector.

10. The system of claim 9, wherein the processor circuit is further configured to:
detect an electrical conductance along the third conductive pathway; and
transmit the data to the ultrasound probe via the second conductive pathway only after detecting the electrical conductance along the first conductive pathway and the third conductive pathway.

11. The system of claim 2,
wherein the processor circuit is further configured for communication with the ultrasound probe via a third conductive pathway,
wherein the processor circuit is further configured to:
prior to detecting the electrical conductance along the first conductive pathway:
detect an electrical conductance along the second conductive pathway;
detect an electrical conductance along the third conductive pathway; and
determine a first time between the detection of the electrical conductance along the second conductive pathway and the detection of the electrical conductance along the third conductive pathway; and
output an alert in response to a second time between the detection of the electrical conductance along a third conductive pathway and the detection of the electrical conductance along the first conductive pathway exceeding the first time.

12. The system of claim 11,
wherein the third conductive pathway comprises a third conductive member of the first connector and a third conductive member of the second connector,
wherein the first conductive member of the first connector comprises a first length, the second conductive member of the first connector comprises a second length, and the third conductive member of the first connector comprises a third length,
wherein the second length is greater than the third length, and
wherein the third length is greater than the first length.

13. The system of claim 1, wherein the processor circuit is configured to monitor the electrical conductance along the first conductive pathway in response to detecting an electrical conductance along the second conductive pathway.

14. The system of claim 13, wherein the processor circuit is further configured to:
determine a time between the detection of the electrical conductance along the first conductive pathway and the detection of an electrical conductance along the second conductive pathway; and
output an alert if the time exceeds a threshold.

15. The system of claim 1, further comprising:
a cable extending between the ultrasound probe and the first connector; and
a console comprising the processor circuit and the second connector.

16. The system of claim 1, further comprising:
a console comprising the processor circuit;
a first cable extending between the ultrasound probe and the first connector; and
a second cable extending between the console and the second connector.

17. The system of claim 1, wherein the ultrasound probe further comprises an integrated circuit in communication with the ultrasound transducer array, wherein the processor circuit is configured to transmit the data along the second conductive pathway to the integrated circuit.

18. An ultrasound system, comprising:
an ultrasound probe comprising an ultrasound transducer array;
a first connector electrically coupled to the ultrasound probe, wherein the first connector comprises:
a first connector body having a first end portion;
a first conductive member coupled to the first connector body and spaced from the first end portion by a first distance; and
a second conductive member coupled to the first connector body and spaced from the first end portion by a second distance different than the first distance; and
a second connector configured for mechanical and electrical coupling to the first connector, wherein the second connector comprises:
a second connector body having a second end portion;
a third conductive member coupled to the second connector body and spaced from the second end portion by a third distance, wherein the third conductive member is configured to be electrically coupled to the first conductive member of the first connector; and
a fourth conductive member coupled to the second connector body and spaced from the second end portion by a fourth distance different than the third distance, wherein the fourth conductive member is configured to be electrically coupled to the second conductive member of the first connector;
wherein electrical coupling of the first conductive member of the first connector and the third conductive member of the second connector indicates that the second conductive member and the fourth conductive member are coupled electrically.

19. The system of claim 18, wherein a first length of the first conductive member of the first connector and a second length of the second conductive member of the first connector are different.

20. The system of claim 18, wherein the first connector further comprises an impedance element electrically coupled to the first conductive member.
